(19) United States
(12) Patent Application Publication
(10) Pub. No.: US 2012/0157742 A1
(43) Pub. Date: Jun. 21, 2012

(54) PRODUCTION OF RENEWABLE FUELS

(75) Inventors: Yun Bao, Bartlesville, OK (US); Dhananjay B. Ghonasgi, Bartlesville, OK (US); Joseph E. Bares, Bartlesville, OK (US); Sundararajan Uppili, Bartlesville, OK (US); Jianhua Yao, Bartlesville, OK (US); Xiaochun Xu, Bartlesville, OK (US); Edward L. Sughrue, Bartlesville, OK (US); Lisa L. Myers, Bartlesville, OK (US)

(73) Assignee: CONOCOPHILLIPS COMPANY, Houston, TX (US)

(21) Appl. No.: 13/332,000

(22) Filed: Dec. 20, 2011

Related U.S. Application Data
(60) Provisional application No. 61/576,618, filed on Dec. 16, 2011.
(60) Provisional application No. 61/424,896, filed on Dec. 20, 2010.

(51) Int. Cl. C07C 7/148

(52) U.S. Cl. 585/703

(57) ABSTRACT

The present disclosure relates to a process for the conversion of oxygen-containing hydrocarbons into long-chain hydrocarbons suitable for use as a fuel. These hydrocarbons may be derived from biomass, and may optionally be mixed with petroleum-derived hydrocarbons prior to conversion. The process utilizes a catalyst comprising Ni and Mo to convert a mixture comprising oxygenated hydrocarbons into product hydrocarbons containing from ten to thirty carbons. Hydro-conversion can be performed at a significantly lower temperature than is required for when utilizing a hydrotreating catalyst comprising Co and Mo (CoMo), while still effectively removing sulfur compounds (via hydrodesulfurization) to a level of 10 ppm (by weight) or less.

### CROSS-REFERENCE TO RELATED APPLICATIONS

This application is a non-provisional application which claims the benefit of and priority to U.S. Provisional Application Ser. No. 61/424,896 filed Dec. 20, 2010 and U.S. Provisional Application Ser. No. 61/576,618 filed Dec. 16, 2011, entitled “Production of Renewable Fuels”, both of which are hereby incorporated by reference.

### STATEMENT REGARDING FEDERALLY SPONSORED RESEARCH OR DEVELOPMENT

None.

### FIELD OF THE DISCLOSURE

The present invention relates generally to a hydrotreating process for converting biologically-derived oils and fatty materials (including triglycerides, diglycerides, monoglycerides, and free fatty acids) into hydrocarbon compounds, especially diesel fuel range hydrocarbons.

### BACKGROUND

As the demand for hydrocarbon fuels increases, the incentives for developing renewable hydrocarbon sources increase as well. Various economic, environmental and political pressures are driving the development of alternative energy sources that are compatible with existing technologies and infrastructure. The development of renewable hydrocarbon fuel sources, such as plant and animal sources has been proposed as a solution to this problem.

One possible alternative source of hydrocarbons for producing fuels and chemicals is the natural carbon found in plants and animals; such as for example, oils and fats. These so-called “natural” carbon resources (or renewable hydrocarbons) are widely available, and remain a target alternative source for the production of hydrocarbons.

Unmodified vegetable oils and fats have also been used as additives in diesel fuel to lower cost and improve the lubricity of the fuel. Processes for converting biomass-derived oils and fats into fuel-range hydrocarbons have been developed. One process is the production of biodiesel, which may be produced by subjecting a bio-derived oil to a trans-esterification process using methanol in order to convert the oil to long-chain esters that comprise biodiesel. After processing, the products produced have combustion properties that are quite similar to petroleum-derived hydrocarbons. However, problems such as injector coking and the degradation of combustion chamber conditions have been associated with these unmodified additives. Moreover, the use of biodiesel as an alternative fuel has yet to be proven cost-effective, due in part to its poor oxidative stability, propensity to gel in cold climates and form gums, as well as its production cost.

One proposed solution to these problems has been to mix biomass-derived fats and oils with petroleum-derived hydrocarbons, then convert the mixture to diesel boiling-range hydrocarbons (mainly hydrocarbon paraffins) via contact with a hydrotreating catalyst to convert oils/fats to renewable fuel. Oxygen atoms in oils/fats are removed by either hydrodeoxygenation to form H₂O or decarboxylation to form carbon monoxide (CO) and carbon dioxide (CO₂) to produce a stable fuel with improved energy content. Olefins are also saturated during this hydrotreating (or hydro-conversion) process, and heteroatoms such as nitrogen and sulfur are removed. Another potential solution is to find a hydro-conversion process that allows solely biomass-derived fats and oils to be converted to diesel boiling-range hydrocarbons without the problems inherent in conventional processes (as mentioned above).

Petroleum-derived hydrocarbons typically contain a greater quantity of sulfur compounds than biomass-derived hydrocarbons, and these sulfur compounds must be removed below a certain level in order to meet regulatory standards. Typically, this is achieved by hydro-conversion using a catalyst comprising cobalt (Co) and molybdenum (Mo), but not nickel (Ni). However, when petroleum-derived hydrocarbons are mixed with biomass-derived hydrocarbons, the high level of oxygen-containing compounds commonly found in biomass-derived hydrocarbons component of the mixture can inhibit catalytic hydrodesulfurization (HDS) of the sulfur-containing compounds commonly present in the petroleum-derived hydrocarbon component. This often leads to increased levels of sulfur-containing compounds remaining in the converted hydrocarbon product that exceeds regulatory standards for diesel fuels.

Some have attempted to overcome this inhibition of HDS by implementing multi-step processes that require multiple reactors and catalyst beds (US2009/0107033, US2008/0156694). A drawback to multiple reactor and catalyst bed processes is that they are expensive, requiring additional capital expenditure to construct, and are also less efficient to operate than a conventional hydrotreating process. Thus, a need exists for an improved single-stage process that can convert mixtures comprising 1) biomass-derived hydrocarbons and petroleum-derived hydrocarbons, or 2) solely biomass-derived hydrocarbons to a fuel in the diesel fuel boiling range that requires a minimal number of steps and maximizes catalyst lifespan to minimize reactor maintenance and cost.

### BRIEF SUMMARY

Accordingly, certain embodiments provide a single-step hydrotreating process for the conversion of oxygen-containing hydrocarbons (preferably, biomass-derived hydrocarbons) that allows a lower conversion temperature to be utilized relative to conventional hydrotreating over a CoMo catalyst. Lower reactor temperature has multiple benefits, including increased efficiency, and increased catalyst lifespan due to a decreased rate of coke deposition. In certain alternative embodiments, a single-step process is provided for the conversion of a liquid mixture of biomass-derived hydrocarbons and petroleum-derived hydrocarbons to produce hydrocarbon compounds suitable for use as a fuel.

Utilizing a feedstock containing solely biomass-derived hydrocarbons, the hydroconversion process of the current disclosure can be conducted at a temperature between about 36° F. (20° C.) to about 90° F. (50° C.) lower than a typical hydroconversion process that utilizes a CoMo catalyst. When utilizing a feedstock comprising a mixture of both biomass-derived hydrocarbons and petroleum-derived hydrocarbons, the hydroconversion process can be conducted at a temperature between about 5° C. to about 15° C. lower than a typical hydroconversion process that utilizes a catalyst that comprises Co and Mo, but not Ni (hereinafter, CoMo). An important consequence of this finding is that the conversion processes disclosed herein are substantially more efficient, meeting regulatory specifications for sulfur removal with less energy input required, and less catalyst coking, leading to longer catalyst life.

Certain embodiments of the process comprise providing a liquid mixture comprising solely biomass-derived hydrocarbons, and contacting the mixture with a catalyst in a reactor under conditions of temperature and pressure that cause conversion of greater than 95% (by vol.) of the mixture, preferably greater than 98% (by vol.), to a product comprising hydrocarbons between C₁₀ and C₃₀ in length, preferably C₁₃ to C₂₀ in length, that are suitable for use as a fuel. The catalyst utilized for this process comprises the metals Ni and Mo, but not Co (hereinafter, NiMo). In certain embodiments, the catalyst concurrently removes sulfur compounds via hydrodesulfurization to a level of about 10 ppm (by weight) or less, where the temperature required for the conversion is at least 36° F. (20° C.) lower, preferably at least 54° F. (30° C.) lower, most preferably at least 72° F. (40° C.) lower than the temperature that would be required to convert the mixture if the catalyst instead comprised Co and Mo, but not Ni. In certain embodiments, the hydrodesulfurization activity of the catalyst is less inhibited by oxygen-containing compounds present in the biomass-derived hydrocarbons than if the catalyst instead comprised Co and Mo, but not Ni.

Optionally, the liquid mixture may comprise mixtures of both biomass-derived and petroleum-derived hydrocarbons. In certain embodiments, the biomass-derived hydrocarbons comprises greater than 10% (by vol.) of the mixture. In those embodiments where the liquid mixture comprises a mixture of both biomass derived and petroleum-derived hydrocarbons, the temperature required to hydroconvert the liquid mixture is at least 5° C. lower (but preferably, at least 10° C. lower) than would be required to hydroconvert the mixture with a catalyst comprising CoMo. In certain embodiments, the hydrodesulfurization activity of the catalyst is less inhibited by the presence of biomass derived hydrocarbons in the mixture than when the catalyst comprises CoMo.

### DETAILED DESCRIPTION

Generally, catalysts comprising the metals Co and Mo, but not Ni (CoMo) have a better hydrodesulfurization (HDS) activity than catalysts comprising Ni and Mo, but not Co (NiMo) in conventional hydrotreating reactors that exclusively treat petroleum-derived hydrocarbon streams. However, the processes disclosed herein build upon our unexpected finding that the HDS activity of NiMo catalysts is far more resistant to inhibition in the presence of a feedstock comprising a large quantity of oxygen-containing molecules (such as, for example, biomass-derived hydrocarbons). In the presence of such feedstocks, we have found that NiMo catalysts have better HDS activity than CoMo catalysts at similar conditions of temperature and pressure. Thus, NiMo catalysts can efficiently convert biomass-derived hydrocarbon feedstocks into products suitable for use as motor fuels at a significantly lower reactor temperature than when using CoMo catalysts, while also maintaining effective HDS activity. This increases the overall efficiency of the hydro-conversion process and also prolongs catalyst lifespan by reducing coke deposition on the catalyst.

In certain embodiments, the present invention provides a hydrotreating process for converting mixtures of oils and fats (triglycerides, or fatty acids of triglycerides) and petroleum-derived hydrocarbons into C₁₀ to C₃₀ hydrocarbon compounds, especially hydrocarbons ranging from C₁₅ to C₁₈ in length that are in the middle distillate boiling-range. The process allows efficient conversion of these mixtures under lower temperature conditions than are required by a typical hydrotreating process that utilizes a catalyst comprising cobalt and molybdenum. An additional benefit of this decreased hydrotreating temperature is increased catalyst lifespan.

According to certain embodiments of the processes disclosed herein, biomass-derived hydrocarbons, such as triglycerides and/or mixtures of triglyceride, are mixed with a petroleum-based hydrocarbon, then contacted with a catalyst composition under conditions sufficient to produce a reaction product comprising hydrocarbons in the middle distillate boiling-range. The amount of biomass-derived hydrocarbons used as the starting material in the present invention may vary depending on the size of the commercial process or suitability of the mixing/reaction vessel. In certain embodiments, the biomass-derived hydrocarbons may comprise from about 1% to about 100% of the total weight of the mixture. In other embodiments, the biomass-derived hydrocarbons may comprise from about 10% to about 50% of the total weight of the mixture.

Dilution of biomass-derived hydrocarbons with petroleum-based hydrocarbons serves to decrease the total oxygen in the feed that must be removed by hydrodeoxygenation (HDO). The petroleum-based hydrocarbons utilized for the process boil at a temperature between about 80° F. (27° C.) to about 1300° F. (704° C.) (90% True Boiling Point). Suitable hydrocarbons may include, for example, middle distillates, which generally contain hydrocarbons that boil in the range from about 300° F. (148° C.) to about 750° F. (399° C.). Examples of middle distillates include, but are not limited to: jet fuel, kerosene, diesel fuel, light cycle oil, atmospheric gas oil, and vacuum gas oil. If a middle distillate is employed in the processes described herein, it generally may contain a mixture of hydrocarbons having a boiling range (ASTM D86) of from about 300° F. (282° C.) to about 750° F. (399° C.). In certain embodiments, the middle distillate employed in the processes described herein has a boiling range of from about 350° F. (332° C.) to about 725° F. (385° C.).

The middle distillate that may optionally be added prior to processing has a mid-boiling point (ASTM D86) of greater than about 500° F. (260° C.). In certain embodiments, the middle distillate feed has a mid-boiling point of greater than about 550° F. (288° C.). In other embodiments, the middle distillate feed has a mid-boiling point of greater than about 600° F. (316° C.). The middle distillate feed has an API gravity (ASTM D287) of from about 15° API to about 50° API. In addition, middle distillate feeds used in the present invention generally have a minimum flash point (ASTM D93) of greater than about 100° F. (38° C.). In certain embodiments, the middle distillate feed has a minimum flash point of greater than about 90° F. (32° C.). In addition to middle distillates, other suitable petroleum-derived hydrocarbons include, but are not limited to, gasoline, naphtha, and atmospheric tower bottom.

Hydrocarbons useful in the present invention generally may contain a quantity of aromatics, olefins, and sulfur, as well as paraffins and naphthenes. The amount of aromatics in the hydrocarbon generally may be in the range of from about 10% to about 90% by weight of aromatics based on the total weight of the hydrocarbon, but are preferably in the range of from about 20% to about 80% by weight, based on the total weight of the hydrocarbon. The amount of olefins in the hydrocarbon generally may be in an amount of less than about 10% olefins by weight based on the total weight of the hydrocarbon. In one embodiment of the present invention, olefins are present in an amount of less than about 5% by weight. In another embodiment of the present invention, olefins are present in an amount of less than about 2% by weight.

In certain embodiments, the quantity of sulfur in the petroleum-based hydrocarbon feedstock prior to hydro-conversion is generally greater than about 50 parts per million by weight (ppmw). In one embodiment of the present invention, sulfur is present in an amount in the range of from about 100 ppmw to about 50,000 ppmw sulfur. In another embodiment of the present invention, sulfur is present in the range of from about 150 ppmw to 4,000 ppmw. As used herein, the term “sulfur” denotes elemental sulfur, and also any sulfur compounds normally present in a petroleum-based hydrocarbon stream, such as middle distillates. Examples of sulfur compounds which may be removed from a hydrocarbon stream through the practice of the present invention include, but are not limited to, hydrogen sulfide, carbonyl sulfide (COS), carbon disulfide (CS₂), mercaptans (RSH), organic sulfides (R—S—R), organic disulfides (R—S—S—R), thiophene, substituted thiophenes, organic trisulfides, organic tetrasulfides, benzothiophene, alkyl thiophenes, dibenzothiophene, alkyl benzothiophenes, alkyl dibenzothiophenes, and the like, and mixtures thereof as well as heavier molecular weights of the same, wherein each R can be an alkyl, cycloalkyl, or aryl group containing 1 to about 10 carbon atoms.

The hydrotreating catalysts useful with the current invention are generally highly active catalysts that are capable of utilizing hydrogen to accomplish saturation of unsaturated materials, such as aromatic compounds. These catalysts are commonly referred to as hydrotreating catalysts in the art, and such catalysts useful in the present invention are effective in the conversion of triglycerides to saturated hydrocarbons when contacted under suitable reaction conditions. Additionally, catalysts useful with the current invention comprise the metals Ni and Mo. Such catalysts are commercially available from companies such as, for example, Haldor Topsoe, Criterion Catalysts and Technologies, and Albermarle, Inc. Catalysts useful in the present invention may contain metal distributed over the surface of a support in a manner than maximizes the surface area of the metal. Examples of suitable solid support materials for the hydrogenation catalyst includes, but is not limited to, silica, silica-alumina, aluminum oxide (alumina, Al₂O₃), silica-magnesia, silica-titania and acidic zeolites of natural or synthetic origin. The catalyst may be prepared by any method known in the art, including combining the metal with the support using conventional means including but not limited to impregnation, ion-exchange and vapor deposition. Preferably, the catalyst support comprises alumina. The catalyst may optionally be promoted with a halogen, such as fluorine or chlorine, in order to enhance the production of desired hydrocarbons. Fluorine, for example, can be incorporated into or onto the catalyst by impregnating said catalyst with ammonium bi-fluoride. Similar methods for incorporating chlorine are known in the art.

The HDS activity of catalysts useful with the current invention is not significantly inhibited in the presence of oxygenated hydrocarbon feedstock, such as biomass-derived hydrocarbons. For embodiments where the feedstock comprises solely biomass-derived hydrocarbons, the catalyst is preferably able to perform HDS of the feedstock to a level of less than or equal to about 10 ppm of sulfur compounds, while operating at conditions of temperature and pressure that are at least 36° F. (20° C.) lower, preferably at least 54° F. (30° C.) lower, most preferably at least 72° F. (40° C.) lower than the temperature that would be required to convert the mixture if the catalyst instead comprised Co and Mo, but not Ni. For embodiments where the feedstock comprises a mixture of biomass-derived hydrocarbons and petroleum-derived hydrocarbons, the catalyst is preferably able to perform HDS of the feedstock to a level of less than or equal to about 10 ppm of sulfur compounds, while operating at conditions of temperature and pressure that are at least 5° C. lower , preferably at least 10° C. lower, than the temperature that would be required to convert the mixture if the catalyst instead comprised Co and Mo, but not Ni.

The process of the present invention can be carried out in any suitable reaction zone that enables intimate contact of the reactants and control of the operating conditions under a set of reaction conditions that include total pressure, temperature, liquid hourly space velocity, and hydrogen flow rate. The reactants may be added to the reaction chamber in any suitable manner or in any suitable order. The catalyst can be added first to the reactants and thereafter, fed with hydrogen. A reactor comprising either one or more fixed catalytic beds or fluidized catalytic beds can be utilized. One example of a fluidized bed reactor that can be useful in the present invention can be found in U.S. Pat. No. 6,890,877, the entire disclosure of which is herein incorporated by reference. The temperature of the reaction zone within the reactor is maintained generally in the range of from about 482° F. (250° C.) to about 797° F. (425° C.). Preferably, the temperature is maintained in the range of from about 500° F. (260° C.) to about 680° F. (360° C.). Regardless of whether a fixed or fluidized reactor is used, the pressure within the reactor housing the inventive process is generally maintained in the range of from about 100 pounds per square inch gauge (psig) to about 3000 psig, preferably in a range from about 100 psig to about 2000 psig. In a reactor containing one or more fixed catalytic beds, the pressure is kept in a range from about 100 psig to about 750 psig, but preferably, in a range of from about 125 psig to about 500 psig. In a reactor containing one or more fluidized catalytic beds, the pressure is maintained preferably in the range of from about 400 psig to about 750 psig, but preferably, in a range from about 450 to 550 psig. The LHSV is generally in the range of from about 0.1 hr⁻¹ to about 10 hr⁻¹. In certain embodiments of the present invention, the LHSV is in the range of from about 0.5 hr⁻¹ to about 5 hr⁻¹. In other embodiments, the LHSV is in the range of from about 1.5 hr⁻¹ to about 4.0 hr⁻¹. In certain additional embodiments, the LHSV is in the range of from about 1.8 hr⁻¹ to 3.0 hr⁻¹. In certain additional embodiments, the LHSV is in a range from about 0.1 hr⁻¹ to about 0.7 hr⁻¹. In certain embodiments, the processes described herein comprise contacting a mixture of biomass-derived hydrocarbons and petroleum-based hydrocarbons with a hydrogen-containing diluent. In certain other embodiments, the processes described herein comprise contacting a mixture comprising solely biomass-derived hydrocarbons with a hydrogen-containing diluent Generally, the hydrogen-containing diluent contains more than about 25% by volume hydrogen based on the total volume of the hydrogen-containing diluent. Preferably, the hydrogen containing diluent contains more than about 50% by volume hydrogen. More preferably, the hydrogen containing diluent contains more than about 75% by volume hydrogen.

The rate at which the hydrogen-containing diluent is charged to the reaction zone is generally in the range of from about 300 standard cubic feet per barrel (SCF/B) of reactants to about 10,000 SCF/B. In one embodiment of the present invention, the hydrogen-containing diluent is charged to the reaction zone in the range of from about 1,200 SCF/B to about 8,000 SCF/B. In another embodiment of the present invention, the hydrogen-containing diluent is charged to the reaction zone in the range of from about 2,500 SCF/B to about 6,000 SCF/B. In another embodiment of the present invention, the hydrogen-containing diluent is charged to the reaction zone in the range of from about 3,000 SCF/B to 5,000 SCF/B. Generally, the triglyceride-containing material, optional middle distillate fuel, and hydrogen-containing diluent may be simultaneously introduced into the reaction zone via a common inlet port(s). In one embodiment of the present invention, hydrocarbon, triglyceride and hydrogen-containing diluent are combined prior to introduction into the reaction zone, and are thereafter co-fed into the reaction zone. Generally, the hydrogen consumption rate under reaction conditions is proportional to the pressure of the reaction conditions employed. In one embodiment of the present invention, hydrogen may be consumed in an amount up to the amount of hydrogen initially charged to the reaction zone. In another embodiment of the present invention, the amount of hydrogen consumed by the reaction at a pressure of less than about 500 psig is less than the amount of hydrogen consumed in the reaction at a pressure of about 500 psig.

In certain embodiments, sulfur compounds present in the hydrocarbons are removed from the hydrocarbon during the conversion process to hydrocarbons in the diesel fuel boiling range. Generally, hydrocarbon products of the conversion process have a sulfur content that is substantially less than the sulfur content present in the reaction feed. Preferably, the sulfur content of the hydrocarbon product of the conversion process is at least 25% less than the sulfur content present in the reaction feed. More preferably, the sulfur content of the hydrocarbon product is at least 50% less than the sulfur content present in the reaction feed. Most preferably, the sulfur content of the product is at least 75% less than the sulfur content present in the reaction feed and contains about 10 ppm or less of sulfur compounds. The conversion product, in accordance with the present invention, generally comprises gas and liquid fractions containing hydrocarbon products, which include, but are not limited to, diesel boiling-range hydrocarbons. The reaction product generally comprises long chain carbon compounds having 13-20 or more carbon atoms per molecule (C₁₃-C₂₀). Preferably, the conversion product comprises carbon compounds having 15 to 18 or more carbon atoms per molecule (C₁₅-C₁₈). In addition, the reaction product can further comprise by-products of carbon monoxide (CO) and carbon dioxide (CO₂).

The acid content of the hydrocarbon product is measured by the total acid number or “TAN.” The total acid number (TAN), as used herein, is defined as milligrams of potassium hydroxide (KOH) necessary to neutralize the acid in 1 gram of oil and is determined using ASTM test method D 644-95 (Test Method for Neutralization Number by Potentiometric Titration). Generally, the total acid number for a yellow grease feed stock is in the range of greater than about 2 mg/KOH/g. In accordance with the present invention, the total acid number for the hydrocarbon product produced in accordance with the present invention will be less than the TAN of the original feedstock.

The cetane number of the hydrocarbon product is determined using ASTM test method D 613-05. With a light cycle oil (LCO) feedstock, the cetane number is typically less than 28 and may in some instances be less than 26, or less than 24. Generally, the cetane number of the hydrocarbon product produced in accordance with the present invention will have a cetane number greater than that of the original feedstock. The cetane number of the hydrocarbon product can also have a higher cetane number by varying the reaction conditions, including the reactor pressure.

Biomass-derived hydrocarbons useful for the processes disclosed herein include triglycerides or fatty acids of triglycerides (or mixtures thereof) that may be converted in accordance with the present inventive process to form a hydrocarbon mixture useful for liquid fuels and chemicals. Any suitable triglyceride can be used in combination with the petroleum based hydrocarbon to form a feedstock. The term, “triglyceride,” is used generally to refer to any naturally occurring ester of a fatty acid and/or glycerol having the general formula

CH₂(OCOR₁)CH(OCOR₂)CH₂(OCOR₃)

where R₁, R₂ and R₃ are the same or different, and may vary in chain length. Vegetable oils, such as for example, canola and soybean oils contain triglycerides with three fatty acid chains. Useful triglycerides in the present invention include, but are not limited to, triglycerides that may be converted to hydrocarbons when contacted under suitable reaction conditions. Examples of triglycerides useful in the present invention include, but are not limited to, vegetable oils including soybean and corn oil, peanut oil, sunflower seed oil, coconut oil, babassu oil, grape seed oil, poppy seed oil, almond oil, hazelnut oil, walnut oil, olive oil, avocado oil, sesame oil, tall oil, cottonseed oil, palm oil, rice bran oil, canola oil, cocoa butter, shea butter, butyrospermum, wheat germ oil, illipse butter, meadowfoam, seed oil, rapeseed oil, borange seed oil, linseed oil, castor oil, vernoia oil, tung oil, jojoba oil, ongokea oil, jatropha oil, algae oil, yellow grease (for example, as those derived from used cooking oils), and animal fats (such as tallow animal fat, beef fat, and milk fat, and the like and mixtures and combinations thereof). Preferably, the triglyceride is selected from the group consisting of vegetable oil, yellow grease (used restaurant oil), animal fats, and combinations of any two or more thereof

The process of the current disclosure will be better understood with reference to the following non-limiting examples. These examples are intended to be illustrative of specific embodiments of the present invention in order to teach one of ordinary skill in the art how to make and use the invention. These examples are not intended to limit the scope of the invention in any manner.

### EXAMPLE 1

A representative diesel blend with approximately 1000 ppm sulfur was mixed with Soybean oil (as a representative for vegetable oils and animal fats) to obtain a 10% by vol. soybean oil feedstock. Commercial hydrotreating catalysts were obtained that contained either cobalt and molybdenum (Catalyst A) or nickel and molybdenum (Catalyst B, Catalyst C, and Catalyst D). These catalysts were pre-sulfided prior to use utilizing techniques commonly known in the art.

After pre-sulfiding, hydrotreating of the 10% by vol. soybean oil was performed at a pressure of 500 psig, an LHSV of 1.0 hr⁻¹, and an H₂/liquid feed of 2250 SCF/B. The hydrotreating temperature was varied between 536° F. (280° C.) and 707° F. (375° C.) in order to determine the threshold temperature needed to achieve essentially 100% conversion of the 10% (by vol.) soybean oil feedstock using a given catalyst. Each of the catalysts tested demonstrated different threshold temperatures for the complete conversion. Catalyst B, Catalyst C and Catalyst D (all catalysts containing Ni and Mo) showed higher activity and required lower threshold temperatures of 590° F. (280° C.), 554° F. (290° C.) and 536° F. (310° C.), respectively, to completely convert soybean oil as compared to Catalyst A 626° F. (330° C.) (See Table 1).

TABLE 1
Threshold conversion temperatures for essentially complete
deoxygenation of a 10% (by vol.) soybean oil feedstock.

| Catalyst | Temp. Threshold |
|---|---|
| Catalyst A (CoMo) | 626° F. (330° C.) |
| Catalyst B (NiMo) | 590° F. (310° C.) |
| Catalyst C (NiMo) | 554° F. (280° C.) |
| Catalyst D (NiMo) | 536° F. (290° C.) |

### EXAMPLE 2

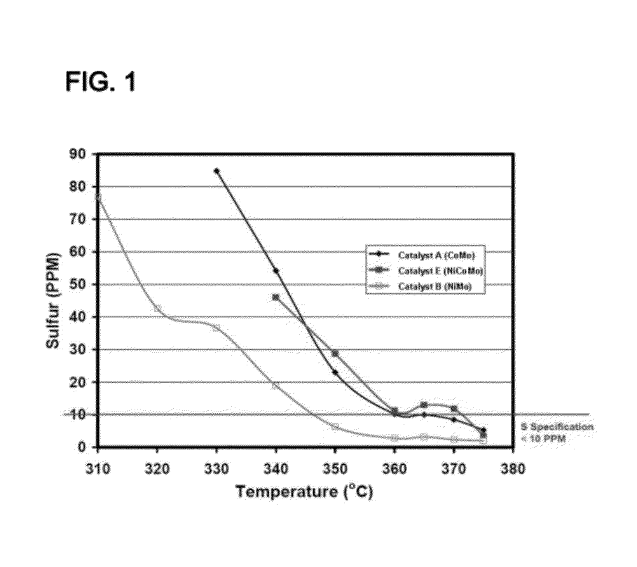

We measured the threshold temperature needed to achieve adequate hydrodesulfurization (HDS) of a bio-derived hydrocarbon mixture sufficient to meet regulatory requirements for ultra low sulfur content (10 ppm or less). Samples were analyzed by ultraviolet light florescence with an Antek Sulfur Express analyzer. In addition to Catalyst A and Catalyst B used in Example 1, a catalyst comprising Ni/Co/Mo (Catalyst E) was tested. Catalysts were pre-sulfided as in Example 1, and conversion of the 10% (by vol.) soybean oil feedstock (prepared as in Example 1) was performed at various temperatures between 536° F. (280° C.) and 707° F. (375° C.). Afterward, the remaining sulfur concentration within each test sample was measured, and the results plotted (see FIG. 1). For all catalysts tested, the temperature required for HDS to 10 ppm (or less) was higher than the threshold temperature needed for complete oil/fat conversion. In feeds containing only petroleum-derived hydrocarbons, CoMo catalysts are typically better at performing HDS than NiMo catalysts. However, in the presence of 10% soybean oil feedstock, we observed that the NiMo-containing catalyst (Catalyst B) achieved adequate HDS (10 ppm) at a significantly lower temperature of approximately 653° F. (345° C.), while Catalyst A or Catalyst E required temperatures of approximately 680° F. (360° C.). This demonstrates that the HDS activity of Catalyst B was less affected by the presence of the biomass-derived hydrocarbons.

Thus, the conversion temperature when utilizing a NiMo catalyst, such as Catalyst B, may be up to 27° F. (15° C.) less than the conversion temperature required by catalysts containing either Catalyst A or Catalyst E, while still removing sulfur compounds to a level that meets government regulations. In embodiments of the process where the feedstock contains little sulfur, such as when using an unmixed biomass-derived hydrocarbon feed, NiMo catalysts offer a significant benefit by allowing the conversion to proceed at a significantly lower temperature 36° F. (20° C.) to 90° F. (50° C.) lower than when using the CoMo Catalyst A, (as shown in Table 1) providing a significant increase in efficiency that also would reduce the rate of catalyst coking, thereby extending the useful lifespan of the catalyst.

### EXAMPLE 3

To better characterize the effect of animal fat addition on the HDS of a feedstock mixture containing both bio-derived hydrocarbons and petroleum-derived hydrocarbons, various amounts of animal tallow were mixed with petroleum-derived diesel feedstocks obtained from several different sources (see Table 2). The reaction conditions utilized for each experiment were: Experiment 1: 660.2 (349° C.), 1025 psig, 0.71 hr⁻¹ LHSV, refinery diesel feed containing 9150 ppm of sulfur compounds; Experiment 2: 640.4° F. (338° C.), 960 psig, 0.66 hr⁻¹ LHSV, refinery diesel feed containing 10,100 ppm of sulfur compounds; Experiment 3: 629.6° F. (332° C.), 588 psig, 0.93 hr⁻¹ LHSV. refinery diesel feed containing 107 ppm of sulfur compounds.

TABLE 2
Catalytic Hydrodesulfurization in the Presence of Increasing Levels
of Bio-derived Hydrocarbons (Tallow)

| | | Temp. | | | Product |
|---|---|---|---|---|---|
| | Catalyst | in ° F. | Feed S | Tallow | S |
| Experiment | (Composition) | (° C.) | (ppm) | (% vol.) | (ppm) |
| 1 | Catalyst A | 660.2 (349) | 9150 | 0.0 | 8.3 |
| | (Co/Mo) | 660.2 (349) | 9150 | 3.0 | 17.4 |
| 2 | Catalyst E | 640.4 (338) | 10100 | 0.0 | 3.4 |
| | (Ni/Mo) | 640.4 (338) | 10100 | 3.0 | 4.2 |
| | | 640.4 (338) | 10100 | 9.0 | 4.6 |
| | | 640.4 (338) | 10100 | 15.0 | 5.2 |
| 3 | Catalyst D | 629.6 (332) | 107 | 0.0 | 12.2 |
| | (Ni/Mo) | 629.6 (332) | 107 | 13.3 | 11.5 |

Table 2 demonstrates that HDS performed by the CoMo Catalyst A was efficient at 660.2° F. (349° C.), but inhibited when 3% (by vol.) of tallow was mixed with the feed. The difference is significant in that the final sulfur levels of the treated 3% tallow mixture do not meet specification for ultra low sulfur diesel. Meanwhile, HDS performed by the NiMo catalysts Catalyst E and Catalyst D was conducted at a temperature that was 19.8° F. (11° C.) and 30.6° F. (17° C.) lower, respectively, yet HDS efficiency was relatively unaffected by the presence of tallow in the feed at concentrations of up to 15% (by vol.).

### EXAMPLE 4

Upgrading of algal oil was performed over hydrotreating catalysts comprising either CoMo on an alumina support, or NiMo on an alumina support. Both catalysts were obtained from a commercial vendor. Generally, catalyst was loaded in a standard ¾ inch diameter reactor with a ¼ inch thermo well and tapped to dense packing with alundum below the catalyst bed. Before upgrading, catalysts were presulfided according to a standard presulfiding procedure. The reaction conditions utilized for conversion of the algal oil feedstock were a pressure of 500 psig, a H2/liquid feed ratio of 6,000 SCF/B, a liquid hourly space velocity (LHSV) ranging from of 0.3 hr⁻¹ to 0.1 hr⁻¹, and a temperature of 680° F. (360° C.). Temperature and LHSV were varied to determine the conditions needed for complete conversion of algae oil. Dimethyl disulfide was added to the algae oil to provide a feed sulfur concentration of 100 ppm to maintain the catalysts in an active, sulfided status. The liquid conversion products were submitted for analysis by HPLC And gas chromatography.

TABLE 2
Conversion of algae oil to biodiesel over CoMo and NiMo catalysts
at different LHSV.

| | CoMo Catalyst | NiMo Catalyst |
|---|---|---|
| LHSV (hr⁻¹) | (% Conversion) | (% Conversion) |
| 0.1 | 100% | not tested |
| 0.2 | 99.6% | not tested |
| 0.3 | not tested | 100% |

### EXAMPLE 5

To confirm the low activity of CoMo catalyst in converting a feedstock comprising solely biomass-derived hydrocarbons, soybean oil was converted over a commercial CoMo catalyst at a LHSV of 0.2 h⁻¹. All other reaction conditions utilized were the same as in Example 4.

When conversion was performed at a temperature of 680° F. (360° C.) HPLC analysis revealed the presence of unconverted soybean oil (indicated by the presence of an emulsion at the oil/aqueous interface). Conversion temperature was increased to 710.6° F. (377° C.), but complete conversion was still not achieved, and was quantified at 97.1% based on HPLC data. Finally, when the LHSV decreased to 0.1 h−1, complete conversion of soybean oil to renewable diesel was confirmed by HPLC analysis. The experimental results confirmed that the activity of this CoMo catalyst is too low to convert 100% vegetable oil at high LHSV.

As used herein, “liquid hourly space velocity” or “LHSV” is defined as the numerical ratio of the rate at which the reactants are charged to the reaction zone in barrels per hour at standard conditions of temperature and pressure (STP) divided by the barrels of catalyst contained in the reaction zone to which the reactants are charged.

As used herein, the term “fluidized catalytic bed” denotes a reactor wherein a fluid feed can be contacted with solid particles in a manner such that the solid particles are at least partly suspended within the reaction zone by the flow of the fluid feed through the reaction zone and the solid particles are substantially free to move about within the reaction zone as driven by the flow of the fluid feed through the reaction zone.

As used herein, the term “fluid” denotes gas, liquid, vapor and combinations thereof.

Although the processes described herein have been described in detail, this description is to be construed as illustrative only and is for the purpose of teaching those skilled in the art the general manner of carrying out the invention. It is to be understood that the forms of the invention shown and described herein are to be taken as examples of embodiments, and that various changes, substitutions, and alterations can be made without departing from the spirit and scope of the invention as defined by the following claims.

### REFERENCES

All of the references cited herein are expressly incorporated by reference. Incorporated references are listed again here for convenience:

1. U.S. Pat. No. 6,890,877 (Meier; Sughrue; Wells; Hausler; Thompson); “Enhanced fluid/solids contacting in a fluidization reactor” (2005).

2. U.S. Pat. App. No. 2009/107033 (Gudde; Townsend); “Hydrogenation Process” (2009).

3. U.S. Pat. App. No. 2008/156694 (Thierry; Karin) ; “Process For The Conversion Of Feedstocks Resulting From Renewable Sources For Producing Gas Oil Fuel Bases With A Low Sulphur Content And With An Improved Cetane Number” (2008).

We claim:

1. A process for producing a fuel, comprising:
a) providing a liquid mixture comprising biomass-derived hydrocarbons;
b) contacting the mixture with a catalyst in a reactor under conditions of temperature and pressure that cause conversion of greater than 98 percent (by vol.) of the mixture to one or more hydrocarbons between C₁₀ and C₃₀ in length that are suitable for use as a renewable fuel,
wherein the catalyst comprises nickel and molybdenum, but not cobalt,
wherein the catalyst concurrently performs hydrodesulfurization on the mixture to remove sulfur compounds to a level of about 10 ppm (by weight) or less,
wherein the temperature required for said conversion is at least 36° F. (20° C.) lower than the temperature that would be required to convert the mixture if the catalyst instead comprised cobalt and molybdenum, but not nickel.

2. The process of claim 1, wherein the temperature required for said conversion is at least 54° F. (30° C.) lower than the temperature that would be required to convert the mixture if the catalyst instead comprised cobalt and molybdenum, but not nickel.

3. The process of claim 1, wherein the temperature required for said conversion is at least 72° F. (40° C.) lower than the temperature that would be required to convert the mixture if the catalyst instead comprised cobalt and molybdenum, but not nickel.

4. The process of claim 1, wherein a majority of the one or more hydrocarbons suitable for use as a renewable fuel comprises one or more hydrocarbons between C₁₃ to C₂₀ in length.

5. The process of claim 1, wherein the mixture comprises solely biomass-derived hydrocarbons.

6. The process claim 1, wherein the pressure is maintained in a range from about 100 psig to about 3000 psig.

7. The process claim 1, wherein the pressure is maintained in a range from about 100 psig to about 2000 psig.

8. A process for producing a fuel, comprising:
a) providing a liquid mixture comprising biomass-derived hydrocarbons and petroleum-derived hydrocarbons;
b) contacting the mixture with a catalyst in a reactor under conditions of temperature and pressure that cause conversion of greater than 98% (by vol.) of the mixture to a product comprising hydrocarbons between C₁₀ and C₃₀ in length that are suitable for use as a fuel,
wherein the catalyst comprises nickel and molybdenum, but not cobalt,
wherein the catalyst concurrently removes sulfur compounds via hydrodesulfurization to a level of about 10 ppm (by weight) or less,
wherein the hydrodesulfurization activity of the catalyst is less inhibited by oxygen-containing compounds present in the mixture than if the catalyst instead comprised cobalt and molybdenum, but not nickel,
wherein the temperature required for said conversion is at least 5° F. (3° C.) lower than the temperature that would be required to convert the mixture if the catalyst instead comprised cobalt and molybdenum, but not nickel.

9. The process of claim 5, wherein the temperature required for said conversion is at least 10° F. (6° C.) lower than the temperature that would be required to convert the mixture if the catalyst instead comprised cobalt and molybdenum, but not nickel,

10. The process of claim 5, wherein a majority of the one or more hydrocarbons suitable for use as a renewable fuel comprises one or more hydrocarbons between C₁₃ to C₂₀ in length.

11. The process of claim 5, wherein the biomass derived hydrocarbons comprise greater than about 10% (by vol.) of the mixture.

12. The process of claim 5, wherein the temperature within the reactor is maintained at about 310° C. or less.

13. The process of claim 5, wherein the pressure is maintained in a range from about 100 psig to about 3000 psig.

14. The process of claim 5, wherein the pressure is maintained in a range from about 100 psig to about 2000 psig.
